(19) United States
(12) Patent Application Publication
(10) Pub. No.: US 2016/0022594 A1
(43) Pub. Date: Jan. 28, 2016

(54) SOFT GEL ENCAPSULATION

(71) Applicant: Russell JAFFE, Vienna, VA (US)

(72) Inventor: Russell Jaffe, Vienna, VA (US)

(21) Appl. No.: 14/775,780

(22) PCT Filed: Mar. 11, 2014
(86) PCT No.: PCT/US14/22993

§ 371 Date: Sep. 14, 2015

Related U.S. Application Data
(60) Provisional application No. 61/790,051, filed on Mar. 15, 2013.

(51) Int. Cl. A61K 9/48; A61K 33/06

(52) U.S. Cl. CPC A61K 9/4833; A61K 33/06; A61K 9/4825

(57) ABSTRACT

A stable soft gel dosage form for micronized magnesium complexes is provided wherein soft gel capsules containing various magnesium complexes can be stored for prolonged periods under typical home storage conditions with better than 98% or 99% ASA integrity and with negligible physical or chemical deterioration of the soft gelatin capsule over an extended period of time. The stable soft gel capsule is sealed at a selected temperature forming a seam in which the gelatin promotes cross-linking across the heat seam. This method of sealing the soft gel provides for a better seal that cures the weeping problem that can result from the prior methods of heat sealing soft gel capsules.

### CROSS REFERENCES TO RELATED APPLICATIONS

This application claims the benefit of U.S. Provisional Patent Application Ser. No. 61/790,051, SOFT GEL ENCAPSULATION, filed on Mar. 15, 2013, the contents of which are incorporated herein by reference in their entirety.

### FIELD

The present disclosure relates to soft gel capsules for use in administering pharmaceuticals or nutritional supplements. More particularly, the present disclosure relates to methods of making soft gel capsules with superior stability when anhydrous ingredients are used.

### BACKGROUND

Oral dosage forms, such as, tablets, pills, chewable tablets, and gel capsules are used to administer various pharmaceutical products. Soft gelatin encapsulation of a solution or suspension of an active pharmaceutical or other fill agent dissolved or suspended in a medium offers many advantages over other dosage forms, such as compressed, coated or uncoated solid tablets, hard dried filled capsules, or bulk liquid preparations. Gelatin encapsulation of a solution or suspension permits more accurate delivery of a unit dose. Such uniformity is more difficult to achieve via a tableting process wherein solids must be uniformly mixed and compressed, or via incorporation of the total dose of active ingredient into a bulk liquid carrier which must be measured out prior to each oral administration. Soft gelatin capsules are also often more stable, more easily transported by patients than bulk liquids, and more convenient since only the required number of doses need be removed from the package.

Soft gelatin capsules are favored by many people for oral administration of medications, at least in part because of their ease of swallowing, their masking of taste and protecting unstable ingredients from oxidative or other damage. The soft gelatin shell makes the capsule easier to swallow, especially for many elderly or infirm, than a tablet or a hard capsule. Furthermore, it is thought that the contents of a soft gel capsule may be potentially more bioavailable following ingestion due to rapid rupture of the capsule and release of the contents in the digestive tract. See, for example, U.S. Pat. Nos. 4,597,885; 4,708,834; 4,795,642; and 4,935,243 by the inventor herein.

Because of the elegance of soft gel capsule, their ability to deliver liquid and suspension formulations in a unit dose and their ease of swallowing, for certain formulations gel capsules are preferred. For example, there are numerous pharmaceuticals and nutritional supplements on the market today that are encapsulated in a soft gelatin capsule. In general, the gelatin of the capsule is a mixture of water-soluble proteins produced by collagen tissue hydrolysis and gelatin extraction. The gelatin is then formed into sheets, and two sheets are sealed together by heat sealing with a pharmaceutical or nutritional supplement disposed between the sheets forming a capsule.

Some supplements may be useful in human health and nutrition. For example, magnesium is an important mineral in nutrition. As part of adenosine triphosphate (ATP), magnesium is used for all biosynthetic processes, glycolysis, formation of cyclic adenosine monophosphate (cyclic AMP), is involved in energy metabolism and energy dependent membrane transport, and is used for ribonucleic acid (RNA) synthesis and transmission of the genetic code.

Magnesium, as an example of divalent cations, is involved in excess of 10,000 enzyme (cell catalyst) actions. Magnesium is especially important to enzymes concerned with oxidative phosphorylation. Magnesium is also an important component of both intracellular and extracellular fluids. Intracellular magnesium is believed to control cellular metabolism by modulating the activity of rate limiting enzymes. Extracellular magnesium is important to the maintenance of electrical potentials of nerve and muscle membranes and for transmission of impulses across neuromuscular junctions. Magnesium has been further understood to be important in maintaining the homeostasis of cardiac and smooth muscle tissues.

Considering that magnesium is an activator for so many important body functions, it is not surprising that its deficiency can lead to a variety of serious physical and mental problems. Health conditions such as muscle spasms and tremors are associated with magnesium deficiency. In addition, nerve irritability, mood instability, high blood pressure (essential, otherwise unexplained), angina (chest pain on exertion), heart arrhythmias (magnesium is ‘nature's calcium channel blocker’) calcium loss/osteoporosis risk and insomnia are also associated with magnesium deficiency. Toxic metals (lead, mercury, cadmium, arsenic, and nickel) can accumulate more rapidly when magnesium stores are low and the replacement of magnesium in the body hastens elimination of toxic metals from the body.

Magnesium is inorganic and is not produced by the human body. Humans must rely upon dietary sources to provide the body with its magnesium requirements. Dietary magnesium intake has been declining in the United States, with a per capita decline of magnesium in the U.S. food supply (estimated as food flowing through the food distribution system) of from 408 mg/day in 1909 to 329 mg/day in 1986, almost a 20% decline. This decline has been attributed to the increase in consumption of processed foods. For example, ‘white foods’ such as refined flour, sugar, fat and processed or synthesized foods contain relatively little magnesium.

In those with normal digestion and assimilation, magnesium absorption from food is believed to be from approximately 40 to 60% of that ingested. However, there are many factors that can inhibit the body's ability to absorb magnesium. For example, phosphoric acid, which is present in most soft drinks, and oxalates in foods combine with magnesium in the intestines and form insoluble compounds that are not absorbed by the body. Other factors that can reduce the function of the magnesium uptake system include: toxic minerals such as lead, mercury, arsenic, cadmium, and nickel; biocidal hormone mimics; metabolic cellular acidosis; phytates in ingested foods; caffeine intake; alcohol consumption; certain medications such as steroids and oral contraceptives; distress; enteropathy and other intestinal disorders; and maldigestion and digestive diseases.

Unfortunately, oral intakes of magnesium are difficult for the body to absorb. It is believed that only 3 to 12% of elemental magnesium, typically in the form of magnesium oxide, magnesium sulfate, magnesium carbonate, or other magnesium salt is absorbed for use by the body. Attempts have been made to make complexes to enhance the absorption of magnesium in the human body. For example, U.S. Pat. No. 8,017,160 describes a two part system in which magnesium salts are administered along with choline citrate. However, this system has the drawback in that it is a two part system and not a one part system or single dosage form. Unfortunately, this two part system creates patient compliance issues and stability problems that shorten shelf life. It would therefore be desirable to formulate a magnesium supplement in a single dosage form that would address the deficiencies noted above.

### SUMMARY

A single dosage form, for example, a soft gel capsule is disclosed herein. In an illustrative embodiment, the soft gel is sealed forming a seam in which the gelatin is cross-linked across the seam. This method of sealing the soft gel provides for a better seal that cures the weeping problem that can result from the prior methods of heat sealing soft gel capsules.

The prior methods of heat sealing are generally performed at temperatures in the range of about 65° C. to about 70° C. This heat sealing forms a seam or seal extending around the capsule. Unfortunately, this heat sealing process may result in a seal that cannot adequately restrain some small molecules and/or compounds resulting in weeping. For example, if anhydrous ingredients draw water and/or glycerol from the gelatin shell, the seam may rupture. In addition, the fill of soft gels can migrate into the gelatin shell causing cosmetic discoloration and/or shortening of shelf life and/or adverse interaction between pharmaceutical API and components of the gelatin shell.

In an illustrative embodiment, a method of forming a soft gel dosage form is disclosed. The method may include providing two sheets of a gelatin composition, aligning the two sheets, and sealing edges of the two sheets at a selected temperature range forming a seam having cross-linked gelatin across the seam.

The soft gel may also be cured by drying the soft gel. A fill composition may be disposed between the two sheets prior to completely sealing the edges of the two sheets. The sealing of the edges of the two sheets may include sealing the edges of the two sheets at a selected temperature adapted to allow the formation of the cross-linked gelatin across the seam.

The providing of the two sheets of the gelatin composition may include providing two sheets of the gelatin composition containing one or more of gelatin in an amount of about 65% w/v; glycerol in an amount of about 15% w/v; sorbitol or sorbitan in an amount of about 14% w/v; purified water in an amount of about 5% w/v; and caramel in an amount of about 1% w/v.

In another embodiment, a method of forming a soft gel dosage form is disclosed. The method may include micronizing an anhydrous magnesium powder into a powder having a heterogeneous particle size, suspending the micronized anhydrous magnesium powder in an emulsion forming a fill composition, disposing the fill composition between two sheets of a gelatin composition, and sealing edges of the two sheets together forming a seam having cross-linked gelatin across the seam to form the soft gel.

According to the disclosure, the sealing of the edges of the two sheets may include sealing the edges of the two sheets at a selected temperature adapted to allow the formation of the cross-linked gelatin across the seam. The suspending of the heterogeneous micronized anhydrous magnesium powder in the emulsion may include suspending about 680.8 mg of the micronized anhydrous magnesium powder in the emulsion. The suspending of the micronized anhydrous magnesium powder in the emulsion may include suspending the micronized anhydrous magnesium powder in about 294.4 mg of medium-chain triglycerides and about 225 mg of a phosphatidyl choline solution.

The gelatin composition may include providing the two sheets of the gelatin composition containing: gelatin in an amount of about 264.6 mg, glycerin in an amount of about 69.3 mg, sorbitol or sorbitan in an amount of about 69.3 mg, purified water in an amount of about 60.1 mg, and caramel in an amount of about 10.7 mg.

In another embodiment, a soft gel dosage form is disclosed. The soft gel dosage form may include a soft gel shell including two sheets of a gelatin composition sealed together forming a seam having cross-linked gelatin across the seam, and a fill composition disposed between the two sheets of the gelatin composition including a micronized anhydrous magnesium powder having a heterogeneous particle size.

The two sheets may be sealed together at a selected temperature adapted to allow the formation of the cross-linked gelatin across the seam. The two sheets of gelatin may include: a gelatin, a glycerin, a sorbitol or sorbitan, a purified water, and a caramel color. The fill composition may further include a medium-chain triglyceride, and a phosphatidyl choline.

### BRIEF DESCRIPTION OF THE DRAWINGS

Embodiments of devices, systems, and methods are illustrated in the figures of the accompanying drawings which are meant to be exemplary and not limiting, in which like references are intended to refer to like or corresponding parts, and in which:

FIG. 1 illustrates a block flow diagram of a method of making a soft gel according to aspects of the present disclosure; and

FIG. 2 illustrates a block flow diagram of a method of making a soft gel containing magnesium according to aspects of the present disclosure.

### DETAILED DESCRIPTION

Detailed embodiments of devices, systems, and methods are disclosed herein, however, it is to be understood that the disclosed embodiments are merely exemplary of the devices, systems, and methods, which may be embodied in various forms. Therefore, specific functional details disclosed herein are not to be interpreted as limiting, but merely as a basis for the claims and as a representative basis for teaching one skilled in the art to variously employ the present disclosure.

In an illustrative embodiment, a soft gel capsule and a method of making the soft gel capsule is disclosed herein. In general, the soft gel includes an outer shell and a filling. In an illustrative embodiment, the outer shell is sealed at a selected temperature range forming a seam in which the gelatin is cross-linked across the seam. For example, the outer shell may be sealed at a temperature of about 10° C. to about 60° C., more preferably about 12° C. to about 30° C., and more preferably about 17° C. to about 23° C. (about 62° F. to about 73° F.). In accordance with these temperature range(s), the soft gel shell may be sealed and the cross-linked gelatin seam may be formed using light energy (i.e. photo sealing and/or photocrosslinking) For example, light, such as but not limited to, ultraviolet light and other types of light in the light spectrum may be used to create the cross-linked gelatin seam.

The outer shell may be composed of at least one gelatin, for example, including, but not limited to a gelatin derived from pigskin, a gelatin derived from bovine bone, a gelatin derived from fish gelatin, other animal-derived gelatin, a chemically modified gelatin, a mixture of gelatin, gelatin elastin mixtures, gelatin ground substance mixtures, partially or completely polymerized gelatins and gelatin mixes, polymers of glycine, proline and any third aminoacid as synthetic gelatin and other gelatins, gelatin hydrolysates and gelatin fragments of the type and combinations thereof.

In an illustrative embodiment, the outer shell may include gelatin in an amount of about 10% to about 99%, more particularly from about 75% to about 95%.

The outer shell may also include one or more additional components, for example, including, but not limited to a coloring agent, a flavoring agent, a plasticizer, a modifier, water, an antioxidant, an anti-tack agents, a softening agent, a starch, an opacifying agent, a water binding or releasing agent, a fatty acid or wax, a cation, an anion, glycerol, glycerin, a synthetic analogue with the same functional properties and other components of the type and combinations thereof.

In an illustrative embodiment, the outer shell may include a gelatin mass at approximately 65% w/v, glycerol at approximately 15% w/v, sorbitol or sorbitan or equivalents at approximately 14% w/v, purified water at approximately 5% and caramel at approximately 1%. In one illustrative embodiment, one or more opacifying agents such as, but not limited to, caramel, titanium dioxide, iron oxide, and plant extracts may be optionally present in an amount of about 0.1% to about 10%. In a further illustrative embodiment, the optional opacifying agent is present in about 0.5%-5%.

In another illustrative embodiment, the soft gel may be composed of gelatin and caramel. The gelatin may be kosher certified Gelatin and be present in an amount of about 400 mg (150 bloom, 99%). The caramel may include, but is not limited, to type 1, type 2, type 3 or type 4 from Sethness Caramel Color, Lincolnwood, Ill., and be present in an amount of about 9 mg to about 36 mg (opacity index/color, AP100). The caramel may be any color and any type as known in the art.

In a further illustrative embodiment, the soft gel may be composed of gelatin and caramel and filled with an elemental magnesium composition as shown in Table 1 below:

TABLE 1
Composition of Soft Gel Capsule, 110
mg Elemental Magnesium/Capsule

| | | | Quantity/Capsule | |
|---|---|---|---|---|
| Name of Ingredient | Reference | Function | mg | %w/w |

Fill Composition

| | | | | |
|---|---|---|---|---|
| Magnesium citrate, | USP¹ | Active | 680.60 | 40.66 |
| tribasic, anhydrous, | | | | |
| from Jost Chemical | | | | |
| Co., St. Louis, MO; | | | | |
| USP (micronized) | | | | |
| Medium-chain | NF | Emulsifying agent | 294.40 | 17.59 |
| triglycerides | | | | |
| Phosal ® 75 SA² | NF³ | Solubilizing agent | 225.00 | 13.44 |
| (Phosphatidyl choline | | | | |
| 70%, equivalent to | | | | |
| lecithin derived from | | | | |
| soy source) from | | | | |
| American Lecithin | | | | |
| Company, Oxford, CT | | | | |

Gel Mass Composition

| | | | | |
|---|---|---|---|---|
| Gelatin | NF | Coating agent | 264.60 | 15.81 |
| Sorbitol Sorbitan | NF | Plasticizer | 69.3 | 4.14 |
| Solution | | | | |
| Glycerin, 99.7% | USP | Plasticizer | 69.3 | 4.14 |
| Liquid Caramel Color | In-house | Colorant | 10.7 | 0.64 |
| Purified Water | USP | Diluent | 60.1 | 3.59 |

| | | |
|---|---|---|
| TOTAL | 1674. | 100 |

¹Tested per magnesium citrate USP monograph plus particle size testing per USP <429>
²Composition: 73.9-88.9% lecithin containing ≧91% phosphatidylcholines, soya (CAS No. 97281-47-5), 5-10% ethanol, 5-10% safflower oil, 1-5% sunflower oil mono- and di-glycerides, 0.1-1% soya fatty acids, and <0.1% DL α-tocopherol
³Tested per Lecithin, NF monograph

The flow rate, temperature, humidity, and pressure are controlled to achieve a consistent flow of the fill composition or emulsion such that from about 1 to about 1000 soft gels are produced each minute of operation at consistent size, shape and with a barely visible seam that allows gelatin molecules to cross link between the soft gel layers to produce a single unit from an upper and a lower sheet with a predetermined weight of material to fill the interior.

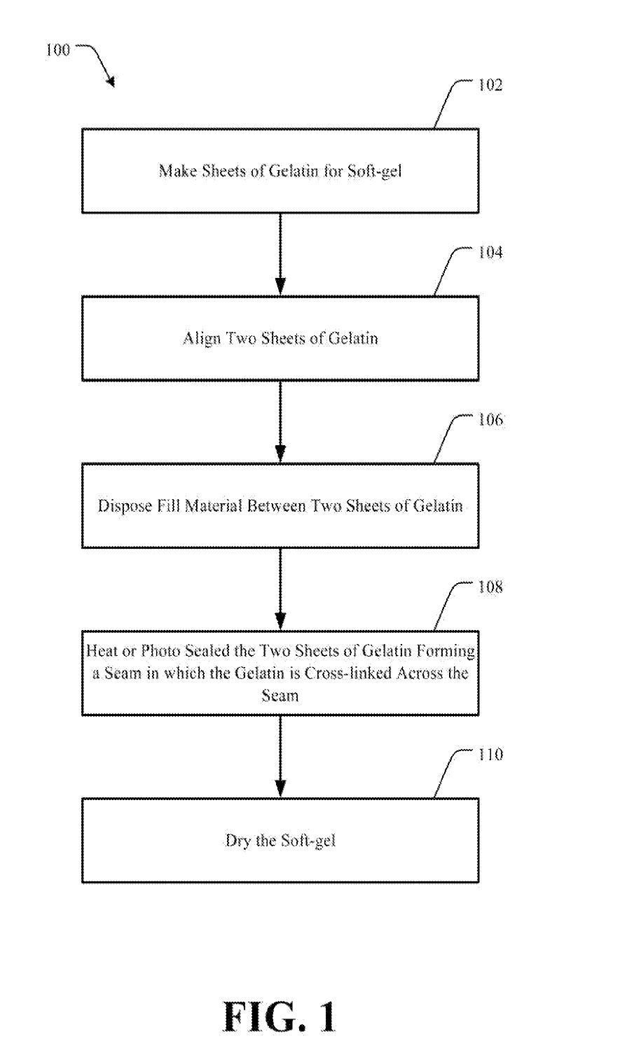

A method 100 of making the soft gel according to an illustrative embodiment is described with reference to FIG. 1. As illustrated in FIG. 1, sheets of gelatin are made, illustrated as block 102, and aligned with one another, illustrated as block 104. The sheets of gelatin may be composed as described herein. A consistent and defined amount of fill material is disposed between two leaves (an upper and a lower sheet) of gelatin, illustrated as block 106, and the edges of the two leaves are heat or photo sealed forming a seam in which the gelatin is cross-linked across the seam, illustrated as block 108. The consistent and defined amount of fill material may include about 1200 mg of fill to about 475 mg of gel mass shell for a total weight of about 1675 mg per soft gel. Soft gels are sized as they are produced. Approximately 95 percent of the soft gels produced should be at the pre-determined size such as a 20 oblong soft gel. Soft gels are then cured or dried according to the art, illustrated as block 110, such as in a drying tunnel and further dried with lint-free cloths.

In a further illustrative embodiment, the filling of the soft gel capsule may be a magnesium complex. The magnesium complex may include but is not limited to a magnesium salt, a quaternary amine or phosphatide and a di-carboxylic acid or tri-carboxylic acid.

The magnesium salt may be any known magnesium salt or a combination of known magnesium salts. Exemplary magnesium salts include but are not limited to magnesium oxide, magnesium glycinate, magnesium ascorbate, magnesium chloride, magnesium sulfate, magnesium orotate, magnesium citrate, magnesium fumarate, magnesium malate, magnesium succinate, magnesium tartrate and magnesium carbonate and anhydrous or structural homologue forms of each of the above.

The magnesium salt may be an anhydrous magnesium salt that is micronized to form a power having heterogeneous particle sizes. The micronization may include milling the anhydrous magnesium salt using a pin mill or other type of milling technique. The particle sizes may be selected to achieve a proper viscous flow rate, proper emulsion, and achieve an effective fill of a smaller volume when the magnesium salt is incorporated into the fill composition. For example, the micronized magnesium salt may have a mean particle size distribution (D50) of about 15 microns to about 35 microns, and a peak particle size of about 80 microns to about 180 microns. Without being bound to any particular theory it is believed that the micronization of the magnesium salt increases the surface area of the magnesium and therefore the bioavailability of the magnesium thereby resulting in an increased uptake of magnesium.

The quaternary amine or phosphatide may include any quaternary amine, phosphatide or a combination of quaternary amines and phosphatides. An exemplary quaternary amine is choline.

The di-carboxylic acid or tri-carboxylic acid may include any di-carboxylic acid, tri-carboxylic acid or combination of di-carboxylic acids and tri-carboxylic acids. An exemplary tri-carboxylic acid is citrate.

The magnesium complex may also include one or more additional components, for example, including, but not limited to water, de-ionized water, glycerol, one or more medium chain triglycerides (MCTs), one or more oils, one or more diluents, one or more lubricants, one or more stearates, one or more disintegrants, one or more fillers, one or more stabilizers, one or more surfactants, and other components of the type and combinations thereof.

Suitable diluents include but are not limited to dicalcium phosphate, calcium sulfate, lactose, cellulose, kaolin, mannitol, sodium chloride, dry starch, and powdered sugar. Lubricants are used to facilitate tablet manufacture; examples of suitable lubricants include but are not limited to magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums, or cross-linked polymers. Fillers include but are not limited to materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers that are now within the pharmaceutical art are used to inhibit or retard drug decomposition reactions that include but are not limited to oxidative reactions. Surfactants may be anionic, cationic, amphoteric, or nonionic surface active agents.

In one embodiment, the filling includes PC-35 (35% phosphatidylcholine), medium-chain triglycerides (MCTs), magnesium citrate, and optionally, water. The PC-35 may be present in an amount of about 380 mg based on an average mw of phosphatides of about 760. The MCTs may be used as a carrier and be present in an amount of about 225 mg. The magnesium citrate may be present in an amount of about 690 mg to provide about 110 mg elemental magnesium based on about 16% elemental magnesium in tri-magnesium citrate, anhydrous API material from Jost Chemical Co., St. Louis, Mo. The water may be deionized water with nitrogen purged and be added as needed.

In this embodiment, the total fill weight of the filling within the soft gel may be about 1300 mg. However, it will be appreciated by those skilled in the art that the total fill weight may be modified based on the size and shape of the soft gel.

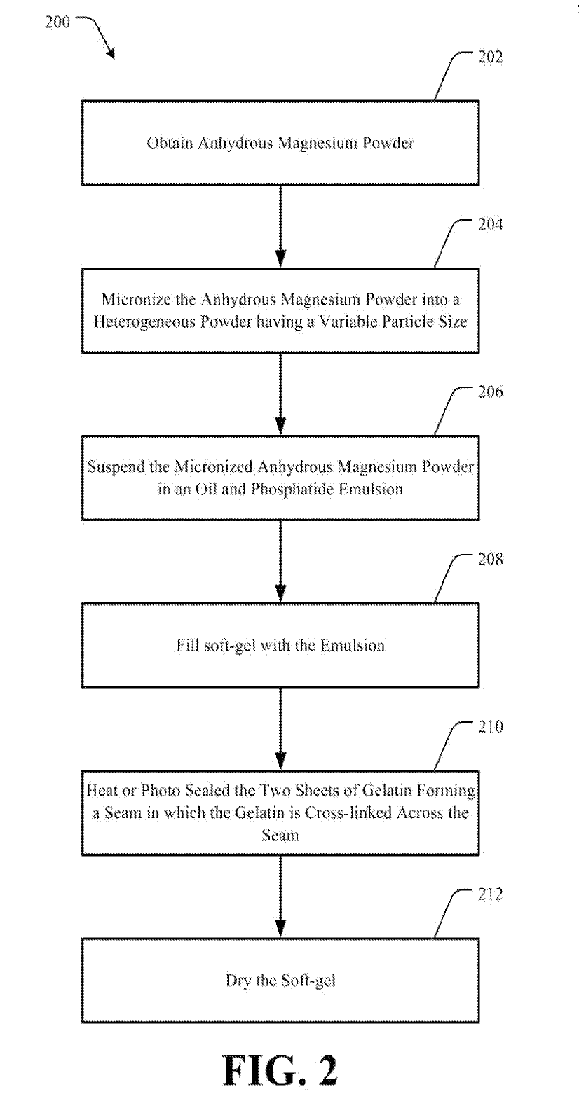

A method 200 of making a soft gel with a magnesium complex filling according to an illustrative embodiment is described with reference to FIG. 2. As illustrated in FIG. 2, an anhydrous magnesium powder, such as tri-magnesium citrate, anhydrous, ultra-fine powder from Jost Chemical Co., St. Louis, Mo., is obtained, illustrated as block 202. The magnesium powder is micronized into a powder having a heterogeneous particle size, illustrated as block 204. The micronized magnesium power is suspended in an oil and phosphatide emulsion, illustrated as block 206. The emulsion is disposed between two leaves (an upper and a lower sheet) of gelatin, illustrated as block 208, and the two leaves are heat or photo sealed forming a seam in which the gelatin is cross-linked across the seam, illustrated as block 210, forming a soft gel. The soft gel is then cured or dried according to the art, illustrated as block 212, such as in a drying tunnel and further dried with lint-free cloths.

The amount of fill may be controlled to allow for potential draw of glycerin and water into the fill from the shell of the soft gel. For example, the anhydrous magnesium salt may act to pull glycerin and water from the shell of the soft gel, thereby increasing the total volume within the soft gel. This increase in volume if not accounted for could cause the soft gel to rupture. Thus, by controlling the volume of fill and accounting for potential draw, the soft gel can accommodate certain increases of pressure/volume without rupturing.

In other embodiments, the filling of the soft gel may be any emulsified mineral supplement. For example, combinations of natural products and synthetic chemicals; anything that tastes bad yet is therapeutic because of being too bitter or other off taste; anything that needs to be protected from light and oxygen; anything that needs to be protected from oxidation; anything that needed precise dosing yet has a liquid or emulsion as an acceptable or preferred delivery system. Any suspension or liquid of sufficient viscosity to flow through the soft gel filling machine may be used.

Although the illustrative embodiments are various magnesium complexes, it will be appreciated by one skilled in the art that the soft gel capsule according to the disclosure can be utilized for any active pharmaceutical ingredient that would benefit from a soft gel encapsulation that prevents weeping of the pharmaceutical formulation. It will also be appreciated by those skilled in the art that the soft gel capsule can be utilized for dietary supplements, vitamin and mineral formulations and naturally derived compositions, such as fish oils and the like.

Although the devices, systems, and methods have been described and illustrated in connection with certain embodiments, many variations and modifications will be evident to those skilled in the art and may be made without departing from the spirit and scope of the disclosure. The discourse is thus not to be limited to the precise details of methodology or construction set forth above as such variations and modification are intended to be included within the scope of the disclosure.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of forming a soft gel dosage form, comprising:
providing two sheets of a gelatin composition;
aligning the two sheets; and
sealing edges of the two sheets together forming a seam having cross-linked gelatin across the seam.

2. The method of claim 1, further comprising curing the soft gel by drying the soft gel.

3. The method of claim 1, further comprising disposing a fill composition between the two sheets prior to completely sealing the edges of the two sheets.

4. The method of claim 1, wherein the sealing of the edges of the two sheets includes sealing the edges of the two sheets at a selected temperature adapted to allow the formation of the cross-linked gelatin across the seam.

5. The method of claim 1, wherein the providing of the two sheets of the gelatin composition includes providing the two sheets of the gelatin composition containing gelatin in an amount of about 65% w/v.

6. The method of claim 5, wherein the providing of the two sheets of the gelatin composition includes providing the two sheets of the gelatin composition containing glycerol in an amount of about 15% w/v.

7. The method of claim 6, wherein the providing of the two sheets of the gelatin composition includes providing the two sheets of the gelatin composition containing sorbitol or sorbitan in an amount of about 14% w/v.

8. The method of claim 7, wherein the providing of the two sheets of the gelatin composition includes providing the two sheets of the gelatin composition containing purified water in an amount of about 5% w/v.

9. The method of claim 8, wherein the providing of the two sheets of the gelatin composition includes providing the two sheets of the gelatin composition containing caramel in an amount of about 1% w/v.

10. A method of forming a soft gel dosage form, comprising:
micronizing an anhydrous magnesium powder into a powder having a heterogeneous particle size;
suspending the micronized anhydrous magnesium powder in an emulsion forming a fill composition;
disposing the fill composition between two sheets of a gelatin composition; and
sealing edges of the two sheets together forming a seam having cross-linked gelatin across the seam to form the soft gel.

11. The method of claim 10, further comprising curing the soft gel by drying the soft gel.

12. The method of claim 10, wherein the sealing of the edges of the two sheets includes sealing the edges of the two sheets at a selected temperature adapted to allow the formation of the cross-linked gelatin across the seam.

13. The method of claim 10, wherein the suspending of the micronized anhydrous magnesium powder in the emulsion includes suspending about 680.8 mg of the micronized anhydrous magnesium powder in the emulsion.

14. The method of claim 10, wherein the suspending of the micronized anhydrous magnesium powder in the emulsion includes suspending the micronized anhydrous magnesium powder in about 294.4 mg of medium-chain triglycerides and about 225 mg of a phosphatidyl choline solution.

15. The method of claim 10, further comprising providing the two sheets of the gelatin composition.

16. The method of claim 15, wherein the providing of the two sheets of the gelatin composition includes providing the two sheets of the gelatin composition containing:
gelatin in an amount of about 264.6 mg;
glycerin in an amount of about 69.3 mg;
sorbitol or sorbitan in an amount of about 69.3 mg;
purified water in an amount of about 60.1 mg; and
caramel in an amount of about 10.7 mg.

17. A soft gel dosage form, comprising:
a soft gel shell including:
two sheets of a gelatin composition sealed together forming a seam having cross-linked gelatin across the seam; and
a fill composition disposed between the two sheets of the gelatin composition including:
a micronized anhydrous magnesium powder having a heterogeneous particle size.

18. The soft gel dosage form of claim 17, wherein the two sheets are sealed together at a selected temperature adapted to allow the formation of the cross-linked gelatin across the seam.

19. The soft gel dosage form of claim 17, wherein the two sheets of gelatin include:
a gelatin;
a glycerin;
a sorbitol or sorbitan;
a purified water; and
a caramel color.

20. The soft gel dosage form of claim 17, wherein the fill composition further includes:
a medium-chain triglyceride; and
a phosphatidyl choline.
